(19) United States
(12) Patent Application Publication
(10) Pub. No.: US 2022/0008888 A1
(43) Pub. Date: Jan. 13, 2022

(54) PRODUCTION OF IMMOBILISED BACTERIOPHAGE

(71) Applicant: FIXED PHAGE LIMITED, Glasgow (GB)

(72) Inventor: Michael Mattey, Glasgow (GB)

(21) Appl. No.: 17/295,414

(22) PCT Filed: Nov. 22, 2019
(86) PCT No.: PCT/EP2019/082323

§ 371 Date: May 19, 2021

(30) Foreign Application Priority Data
Nov. 22, 2018 (EP) 18207889.9

(51) Int. Cl. B01J 19/08; C12N 7/00

(52) U.S. Cl. CPC B01J 19/088; C12N 7/00; B01J 2219/0896; B01J 2219/0886; B01J 2219/0849

(57) ABSTRACT

Bacteriophage is covalently attached to a substrate by (a) combining (i) substrate with (ii) bacteriophage, wherein prior to or during the combining (i) or (ii) or both (i) and (ii) are activated, and wherein (b) during the combining the bacteriophage is contained within a liquid droplet of average diameter 150 microns or less.

### FIELD OF THE INVENTION

The present invention relates to methods and apparatus for immobilisation of bacteriophage onto substrates such as particles, filaments and planar surfaces and to the compositions made thereby. In particular the invention relates to large scale process for immobilisation of bacteriophage, especially onto particles.

### BACKGROUND TO THE INVENTION

In recent years, as resistance to conventional antibiotics has continued to grow and the application of chemical biocides becomes increasingly unacceptable on environmental grounds, attention has turned to alternative methods for control of bacterial contamination. One promising approach involves the application of bacteriophages.

From WO 03/093462 it is known to attach bacteriophage to substrates using chemical methods or an electrical discharge, producing bacteriophage covalently attached e.g. to particles or polymer strips.

In WO 2007/072049 those methods were developed to the use of a pulsed field corona discharge; particles activated by the discharge were dropped into bacteriophage solutions. Similarly, in WO 2012/175749 bacteriophage solutions were combined with activated seeds.

Importantly, bacteriophages immobilised in this way retain their antimicrobial potency and, beneficially, additional stability is conferred so that resistance to degradation and desiccation is significantly enhanced.

Nevertheless a number of problems exist with known methods. In methods described in the prior art, activation of particles by passing them through an electric discharge can result in over heating thereof and/or of adhesion of particles to the apparatus. Control of the number of bacteriophage per particle or the uniformity of attachment over a planar or filamentous substrate is generally not possible, leading to erratic and inconsistent results. Additionally in the case of fine particles (which must be handled as powders) there are no currently available methods for ensuring the surface of particles (comprising fine powders) can be quickly and reliably brought into juxtaposition with bacteriophage so that immobilisation can occur. Decay of free radicals generated using an electric discharge is rapid and existing systems may not bring together substrate and bacteriophage quickly enough for efficient bonding.

In the art, only batch manufacturing methods are practical. For example, materials are placed in a shallow activation vessel and subjected to corona activation from above and then quickly brought into contact with a solution containing the bacteriophages to be immobilised. This method is more or less acceptable for sheet (planar) material with immobilisation occurring at one surface, although it is also used inefficiently for filamentous and particulate material. Self evidently certain aspects of the surface of, say, a spherical particulate may have greater quantities of bacteriophages immobilised than another, whereas a more even distribution may be preferable. A similar effect may be seen in filamentous material of any cross section.

### OBJECTS OF THE INVENTION

One object of the invention is to provide alternative methods for manufacture of compositions comprising bacteriophage covalently immobilised onto substrates. An object of preferred embodiments is to provide improved methods, especially in which bacteriophage is more evenly distributed over the product to which it is attached. A further object of preferred embodiments is to provide new products with bacteriophage attached. A still further object of preferred embodiments is to provide methods that can be used continuously. Devising apparatus to carry out the methods are further objects. Another object of specific embodiments is to provide methods and systems that bring together activated substrate and bacteriophage rapidly, continuously and at large scale.

### SUMMARY OF THE INVENTION

Accordingly, the invention provides a method of covalently attaching a bacteriophage to a substrate, comprising:
a) generating a plasma between two electrodes;
b) flowing a fluid between the electrodes to displace the plasma or a portion of the plasma to a displaced zone not between the electrodes;
c) (i) introducing the substrate into plasma in the displaced zone without the substrate passing between the electrodes and combining the substrate with the bacteriophage, or (ii) introducing the bacteriophage into plasma in the displaced zone without the bacteriophage passing between the electrodes and combining the bacteriophage with the substrate.

Apparatus of the invention, for carrying out such methods, comprises:
a) first and second electrodes and a plasma generator for producing a plasma between the electrodes;
b) a first conduit connected to a supply of fluid under pressure for introducing a fluid flow between the electrodes, wherein flow of the fluid displaces the plasma or a portion of the plasma to a displaced zone located not between the electrodes;
c) a second conduit connected to a supply of bacteriophage or substrate for introducing the bacteriophage or the substrate into the displaced zone without passing between the electrodes;
d) a chamber in which bacteriophage and substrate are combined to covalently attach the bacteriophage to the substrate.

Also provided by the invention is a method of covalently attaching a bacteriophage to a substrate, comprising:
a) combining (i) substrate with (ii) bacteriophage, wherein prior to or during the combining (i) or (ii) or both (i) and (ii) are activated, and wherein
b) during the combining the bacteriophage is contained within a liquid droplet.

Apparatus of the invention, for carrying out such methods, comprises:
a) (i) a supply of substrate and (ii) means to generate droplets containing bacteriophage;
b) (i) a plasma generator for generating a plasma in combination with a droplet activating station to contact the droplets of (a) with the plasma, or (ii) a plasma generator for generating a plasma in combination with a substrate activating station to contact the substrate with the plasma; and
c) a chamber in which substrate and bacteriophage can be combined at the same time as or after contact with the plasma so as to form a covalent bond between the bacteriophage and the substrate.

Still further provided by the invention is a method of covalently attached two bacteriophage of different strain or type to a single substrate particle, comprising:
a) combining the particle with a first bacteriophage, wherein prior to or during the combining the particle or the bacteriophage or both are activated, so as to yield an intermediate product comprising a particle to which the first bacteriophage is covalently attached; and
b) combining the intermediate product with a second bacteriophage of different strain or type to the first, wherein prior to or during the combining the intermediate product or the second bacteriophage or both are activated, so as to yield a product comprising a particle to which the first and second bacteriophage are covalently attached.

### DETAILED DESCRIPTION OF THE INVENTION

A problem addressed by the invention is that of damage to or loss of substrate while being activated by an electric discharge.

The invention accordingly provides a method of covalently attaching a bacteriophage to a substrate, comprising:
generating a plasma between two electrodes;
flowing a fluid between the electrodes to displace the plasma or a portion of the plasma to a displaced zone not between the electrodes;
introducing the substrate or the bacteriophage into plasma in the displaced zone without passing between the electrodes; and
combining the bacteriophage and the substrate.

Hence, substrate or bacteriophage does not pass between the charged electrodes that form the activating field. The risk of excessive heating effects due to passing between electrodes can be reduced or eliminated. Further, the risk of collision with electrodes, leading e.g. to fusing of particles with and melting of particles onto electrodes can be similarly reduced or eliminated. The process is rendered more efficient.

Either bacteriophage or substrate or both can be activated. Hence in one method substrate is introduced into plasma in the displaced zone to activate the substrate and then combined with bacteriophage. Other methods comprise introducing bacteriophage into plasma in the displaced zone to activate the bacteriophage and combining activated bacteriophage with substrate to yield the product—namely, bacteriophage covalently attached to the substrate. Further options are for both bacteriophage and substrate to be introduced into plasma in the displaced zone to activate both and combining activated substrate with activated bacteriophage to yield the product.

The substrate or bacteriophage may pass into the displaced zone to the side of and adjacent the electrodes. The fluid flow may be such that the displaced zone is spaced from the electrodes, further reducing the heating and collision risks associated with prior art methods. By varying the fluid flow, the plasma may be spaced 1 mm or more, 5 mm or more or 10 mm or more from the electrodes—increasing flow further distancing the plasma from the electrodes.

Generally, the electrodes and the displaced zone remain static, and the substrate/bacteriophage are moved through the zone. It is also optional to hold the substrate stationary and, using portable apparatus, move the displaced zone relative to the substrate. Methods may comprise moving the displaced zone across a surface of the substrate, for example scanning apparatus that creates the electric field and the plasma relative to the substrate, which doesn't move. In one particular example, the method comprises applying bacteriophage to substrate surface and moving the displaced zone across the surface of the substrate to which bacteriophage has been applied. The plasma comes into contact with the bacteriophage on the surface, activating one or the other or both, resulting in covalent attachment.

Suitably, the fluid is a gas. This can be blown through a tube, exiting in the vicinity of the electrodes in a moving stream of sufficient velocity to displace the plasma from between the electrodes.

Known methods and means for generating plasma via an electric discharge between the electrodes can be used. A corona discharge can be used and especially a pulsed or pulse field corona discharge.

In particular embodiments of the invention, described below in more detail, the substrate is or comprises particles. These can be flowed or passed continuously through the (displaced) plasma, meaning the invention enables continuous operation. Preferred particle materials and diameters are as described elsewhere herein.

In other embodiments, bacteriophage are introduced into the plasma. Preferably, the bacteriophage are provided as a suspension in liquid droplets. Again, this allows for continuous operation. Suitable droplets have a diameter of 250 microns or less. The method is generally carried out with droplets of volume median diameter 150 microns or less, preferably a volume median diameter of 100 microns or less, more preferably 50 microns or less. Droplet size herein is measured by conventional means, for example using laser diffraction equipment and methods, such as with a Malvern Analyzer (Malvern Instruments Ltd, Worcestershire, United Kingdom).

Also provided by the invention is apparatus for carrying out the methods of the invention. Hence, apparatus of the invention comprises circuitry, electrodes and means to generate a flow of fluid to generate a plasma between the electrodes that can be displaced in whole or part so that particles or bacteriophage can pass through plasma without passing between the electrodes.

A particular apparatus of the invention, set out in more detail below, comprises:
a) first and second electrodes and a plasma generator for producing a plasma between the electrodes;
b) a first conduit connected to a supply of fluid under pressure for introducing a fluid flow between the electrodes, wherein flow of the fluid displaces the plasma or a portion of the plasma to a displaced zone located not between the electrodes;
c) a second conduit connected to a supply of bacteriophage or substrate for introducing the bacteriophage or the substrate into the displaced zone without passing between the electrodes;
d) optionally, a chamber in which bacteriophage and substrate can be combined to covalently attach the bacteriophage to the substrate.

By providing the displaced plasma, the substrate or bacteriophage can be activated while avoiding the risks and disadvantages of having that material pass between the electrodes. To control the location of the displaced zone of plasma, a control mechanism can be included so the flow can be varied, varying the displacement of the plasma.

An option is for the apparatus to comprise a third conduit, the second conduit being for introducing bacteriophage into the displaced zone and the third conduit being for introducing substrate into the displaced zone, so that bacteriophage and substrate are combined in the displaced zone. The apparatus is further optionally adapted in accordance with other optional and preferred embodiments of the methods of the invention.

In embodiments of the apparatus that are portable, and omit the combination chamber, an output of electrically charged particles is provided. This can be directed at bacteriophage, for example bacteriophage droplets which have been applied to a carrier substrate, such as a plainer material or a wound dressing, etc. Thus, the apparatus provides a gun to provide a stream of activated particles to be directed at target bacteriophage.

All embodiments of this aspect of the invention have the advantage that activation of the substrate (e.g. particles) or bacteriophage (e.g. in droplets) avoids passing between electrodes. The optional and preferred features of this aspect of the invention may be used in combination with optional and preferred features of other aspects of the invention as described herein, including as described below.

A further problem addressed in the invention is that attachment of bacteriophage to substrate can be uneven or otherwise difficult to control or difficult to carry out continuously or on a large scale.

Accordingly, the invention provides a method of covalently attaching a bacteriophage to a substrate, comprising:
a) combining (i) substrate with (ii) bacteriophage, wherein prior to or during the combining (i) or (ii) or both (i) and (ii) are activated, and wherein
b) during the combining the bacteriophage is contained within a liquid droplet.

Using droplets, within which bacteriophage are typically suspended, allows improved attachment to the substrate. For example, using a predetermined concentration of bacteriophage (number of bacteriophage per mL of liquid) and droplets of known size or within a given size range allows the number of bacteriophage being combined with substrate to be controlled. In embodiments of the inventions, particles of given size or within a given size range are activated and combined with droplets so as to control the number of bacteriophage per particle, within reasonable boundaries and standard deviations.

Methods of the invention are distinct from known combination of activated particles with solutions of bacteriophage; in typical methods, especially using droplets of bacteriophage and particles, the droplets are of diameter 150 microns or less, such as having a volume median diameter of 150 microns or less, preferably of 100 microns or less, more preferably of 70 microns or less. Separately, the substrate particles are generally of diameter 500 microns or less, suitably having mass median diameters of 500 microns or less, 300 microns or less, suitably 150 microns or less, preferably of 100 microns or less, more preferably of 70 microns or less. In a specific example described below, droplets of about 10 microns diameter were combined with particles of about the same size.

An often-used liquid for the droplets is water; and other aqueous solutions can also be used. In a specific embodiment described below in more detail, a fine mist of an aqueous suspension of bacteriophage was brought into contact with a superabsorbent polymer pre-activated using pulse field corona discharge. Tests confirmed active bacteriophage had been attached to the polymer and weighing of the polymer before and after confirmed only a small weight gain (about 25-30%) through water absorption by the polymer. In other methods, the droplets are liquid that is non-aqueous. In all cases, as will be appreciated, the liquid used should be suitable for the bacteriophage and e.g. non-denaturing or otherwise non-destructive of the bacteriophage—the product of covalent attachment according to the invention comprises infective bacteriophage.

Examples of non-aqueous liquids include compounds and compositions that are gaseous at atmospheric pressures and 20° C., wherein the method is carried out under conditions of temperature and pressure such that the compound forms liquid droplets. Droplets of liquid carbon dioxide are used in certain embodiments to contain the bacteriophage, which are covalently attached to substrate, and the liquid component can then be removed e.g. by adjusting the temperature or the pressure or both.

Thus, the invention comprises methods in which, when using such a liquid droplet, the covalently attached bacteriophage and substrate product is subjected to modified conditions of temperature and/or pressure so that the compound evaporates, yielding dry product.

These methods can be used in selected manufacturing situations, such as for attachment of bacteriophage to a water-sensitive substrate. In a specific embodiment, the substrate comprises a superabsorbent polymer; in this way the methods can be carried out to attach bacteriophage to such substrates while avoiding water or aqueous solutions that would adversely react with (e.g. be absorbed by) and risk damaging or reducing the absorbance of the end product.

Also provided by the invention therefore are compositions comprising superabsorbent polymers to which bacteriophage are covalently attached, which can be made by the invention, wherein the polymer is preferably substantially non-wetted. Examples of compositions include fabric, woven fabrics, diapers, nappies, sanitary towels, sanitary napkins, clothing and undergarments.

All superabsorbent polymers are believed suitable for these compositions, and including both low and high density cross-linked superabsorbent polymers. Polyacrylate-containing polymers, e.g. sodium polyacrylate, and polymers based thereon are suitable. Other suitable such polymers are polyacrylamide copolymers, ethylene maleic anhydride copolymers, cross-linked carboxymethylcelluloses, polyvinyl alcohol copolymers, cross-linked polyethylene oxides, and starch grafted copolymers of polyacrylonitrile.

Advantageously, attachment can be carried out so as to avoid wetting of the polymer. Suitably, the polymer absorbs the liquid of the droplet(s) up to no more than 3 times the polymer weight (bearing in mind the capacity of these polymers, which can be several hundred times the polymer weight), preferably nor more than 1 times the polymer weight, preferably no more than 50% the polymer weight, preferably no more than 25% the polymer weight and very preferably no more than 10% the polymer weight.

In use of the invention, there are various other options. Methods can comprise activating the substrate and combining activated substrate with the liquid droplet, or activating the droplet and combining the activated droplet with the substrate. It is further optional to activate both substrate and droplet and combine them.

As stated elsewhere, the invention allows control of product formation. Hence methods comprise combining droplets of a predetermined size prepared from a suspension of bacteriophage of predetermined concentration so as to control the number and/or density of bacteriophage attached to the substrate. Suitable bacteriophage concentrations are in the range 10⁸-10¹⁰ per mL.

It is preferred that particles and droplets are combined, and that their sizes are controlled so as to be within certain limits. Hence, methods may comprise combining droplets of diameter 150 microns or less with particles of diameter 150 microns or less, preferably combining droplets of volume median diameter 100 microns or less with particles of mass median diameter 100 microns or less. Separately, the method may comprise combining droplets of volume median diameter up to 200 microns or up to 100 microns or up to 20 microns with particles of mass media diameter up to 200 microns or up to 100 microns or up to 20 microns.

It is further preferred that the respective sizes of particles and droplets are controlled. Thus, methods may comprise combining particles and droplets wherein the ratio of the respective diameters (volume mean diameter for droplets and mass median diameter for particles) is from 1:10-10:1, from 1:3-3:1 or from 1:2-2:1. In specific methods carried out in examples, the ratio was approximately 1:1.

In operation of the droplet based invention, it has been found that activated particles generally combine with a limited number of droplets, and the respective sizes and ratios of sizes as described above enables control of properties of the product, especially the number of bacteriophage per particle. Use of droplets containing bacteriophage that are closer in size to the particles tends also to distribute the points of attachment more evenly over the substrate, giving a further enhanced product.

Still further embodiments of the invention, which can be adopted in combination with one or more other embodiments, comprise designing the charges on particles and droplets to promote combination. Methods hence comprise activating both particles and substrate, wherein the particles are activated using an electric discharge and the substrate is activated using an oppositely charged electric discharge.

The discharge is suitably a corona discharge, preferably pulse or pulsed field. Both negative and positive coronas can be used. In a specific embodiment, the particles are activated using a negatively charged corona discharge and the droplets using a positively charged one. In specific methods carried out in examples, polymer particles of size approximately 10 microns activated with a negative pulse field corona discharge were combined with droplets of size approximately 10 microns activated by contact with a positive corona discharge.

Further embodiments comprise comprising simultaneously forming and activating the particles.

Also provided by the invention is apparatus for carrying out the droplet based methods. An apparatus includes a droplet generator to make bacteriophage containing droplets and means to combine the droplets with the substrate. The apparatus can include a station for activating the droplets (e.g. via a corona discharge) or a station for activating substrate (again e.g. via a corona discharge).

A particular apparatus for carrying out the methods comprises:
a) means to generate droplets containing bacteriophage;
b) a plasma generator for generating a plasma in combination with (i) a droplet activating station to contact the droplets with the plasma, or (ii) a substrate activating station to contact the substrate with the plasma; and
c) a chamber in which substrate and bacteriophage can be combined at the same time as or after contact with the plasma so as to form a covalent bond between the bacteriophage and the substrate.

The apparatus of certain embodiments works with droplets e.g. of liquid carbon dioxide. One such apparatus hence is adapted to generate liquid droplets of a solvent or solution that is a gas at room temperature and 20° C.

One apparatus of the invention has a plasma generator adapted for generating a plasma to activate the substrate. Another has a plasma generator adapted for generating a plasma to activate the droplets containing bacteriophage. In other embodiments, the plasma generator and the chamber are arranged so that the bacteriophage and the substrate are combined and contacted with the plasma so as to be activated at the same time and in the same chamber.

Another problem addressed herein is that of including, within products, individual particles that have covalently attached to them bacteriophage of different strains.

By operation of the droplet invention sequentially it is possible to provide a product with two bacteriophages reliably attached. The invention thus further provides a method of covalently attaching two bacteriophage of different strain or type to a single substrate particle, comprising:
a) combining the particle with a first bacteriophage, wherein prior to or during the combining the particle or the bacteriophage or both are activated, so as to yield an intermediate product comprising a particle to which the first bacteriophage is covalently attached; and
b) combining the intermediate product with a second bacteriophage of different strain or type to the first, wherein prior to or during the combining the intermediate product or the second bacteriophage or both are activated, so as to yield a product comprising a particle to which the first and second bacteriophage are covalently attached.

Known methods tended to use a solution of bacteriophage, or a solution containing a mixture of bacteriophages. The resultant product, however, did not reliably provide, say, a particle to which both types of bacteriophage present in the solution were attached.

In further options of the method, the product of these steps is then combined with a third bacteriophage of different strain or type to the first and second, wherein prior to or during the combining the product or the third bacteriophage or both are activated, so as to yield a further product comprising a particle to which the first, second and third bacteriophage are covalently attached.

The present method overcomes the art deficiency, yielding more homogenous products. A composition obtainable using the method comprises a plurality of particles to which first and second bacteriophage of different strain or type are covalently attached, wherein at least 50% of the particles by number comprise at least one first bacteriophage and at least one second bacteriophage.

Generally, at least 60%, preferably at least 70% and more preferably at least 80% of the particles by number comprise at least one first bacteriophage and at least one second bacteriophage.

Optional and preferred embodiments of the invention of the other inventions are described herein can be used in preparation of the products with two or more bacteriophage attached. Further embodiments and features are now described.

Combining Substrate and Phage

In use of one series of examples of the invention, substrate and bacteriophage are brought together within a mixing chamber as a solution, or slurry, or in a vapour or gaseous form. The reactants, either singly or together, are subjected to corona discharge activation prior to entry into the mixing chamber or within the mixing chamber, and are transferred into a chamber suitable for drying and collection of particles. Discharge into the drying chamber may be through atomisation (pneumatic or mechanical) with particles dried e.g. using dry inert gas swirl, and finally collected.

Substrate and bacteriophages are preferably electrically charged such that each carries a charge opposite to the other, causing substrate and bacteriophage to be attracted and distributed relative to one another so that following corona discharge activation bacteriophage are approximately equally distributed upon the surface of the particle.

The invention provides processes for the covalent attachment of bacteriophages to substrate (beads, powders or other particles), wherein during the manufacture of the substrate, such as particles made by electrospray methods, the solvent is evaporated before attachment of phage to particle.

In an example of a method of the invention phage particles are covalently attached to particles of a substrate. This is achieved by entraining particles of a set size in a flowing stream of gas and entraining droplets of liquid of a set size containing phage in a second flowing stream of gas. Treating the entrained particles and/or droplets using corona discharge and combining the gas streams brings the droplets and particles together and causes the phage to become covalently linked to the particles; the phage that are so linked retain their infectivity. After the phage are covalently linked to the particles the particles are collected and, if necessary, dried.

Methods of the invention represent an improvement over previous methods of covalent attachment of phage to substrate particles in respect of the consistency of distribution and frequency with which phage are covalently linked to the particles. Matching the droplet size to substantially match the particle size, e.g. within a diameter ratio of about 4:1 to 1:4, preferably from 1:3 to 3:1, leads to a more even distribution of phage over the surface of the particle. In addition the concentration of phage in the liquid can be set so as to accurately control the number of phage linked to individual particles.

In embodiments of the invention, a stream of gas is used to entrain and carry particles through an apparatus whereby it is treated by exposure to a corona discharge and is thus activated, i.e. the surface of the particle now comprises free radicals that are short lived but highly susceptible to forming covalent bonds with molecules that they come into contact with. A second stream of gas is used to entrain and carry droplets of liquid containing bacteriophage (phage) through an apparatus wherein the droplets are treated by exposure to corona discharge and thus too become activated. The droplets are formed by spraying the phage solution into the entraining gas stream.

The droplets are preferably aqueous but may comprise another liquid such as liquid carbon dioxide or a volatile organic solvent. The gas used to entrain the particles and droplets is preferably air but other gases such as nitrogen, hydrogen or argon may be used.

The two gas streams in which the activated matter is entrained are then combined and the particle and droplet are thereby brought into contact with one another. This contact causes the phage to be covalently linked to the particle. The activation effect of treatment with corona discharge is very short lived and so the matter entrained in the gas streams are suitably combined within 1 second of the corona-discharge treatment.

A positive or a negative corona discharge may be used to treat the particles or the droplets. A positive corona discharge imparts a positive charge to the treated matter and a negative corona-discharge imparts a negative charge. As particles and droplets of similarly charged matter will repel one another, particles and droplets are generally treated using different types of corona discharge. However, all combinations of corona-discharge treatment, including leaving one or other of the particles or droplets untreated, are possible.

Preferably the particles are treated using a negative corona discharge and the droplets are treated using a positive corona discharge; in examples carried out by the inventors this has yielded good results.

Advantages of this pattern of treatments include at least the manner in which the corona-discharge treated particles are electrostatically attracted to the corona-discharge treated droplets yet are repelled by other treated particles and, similarly, the treated droplets are mutually repelled from one another.

The flow rate of the gas entraining the droplets and particles may be adjusted to optimise the efficiency of the activation effect of the corona discharge treatment, and thus the efficiency of attachment of the phage to the particles.

The particles with covalently attached phage are then collected from the gas stream and any remaining liquid from the droplet is removed. Preferably the liquid will have vaporised in the gas stream before collection. A particular advantage of using a volatile solvent for the phage containing droplets is that the drying process is both quicker and is simplified because of the relative ease of being able to dry the treated particles while they are entrained in the gas stream. The path of the gas and its flow rate are suitably adjusted to optimise the process of drying the particles.

A further element of this invention is that particles with phage covalently attached to them can be re-treated one or more times by a similar process in order to covalently attach another type or types of phage to the particle. In this way a particle with an evenly distributed and well-defined population of phage can be produced.

A particular embodiment of the invention uses the following elements:
(i) nozzles or injectors to form two streams of gas in which to entrain particles and droplets of liquid containing phage, each being of a defined size—the flow rate and path of the gas is adjustable to optimise the efficiency of the apparatus;
(ii) apparatus to introduce particles into the stream of gas and treat them with a corona discharge; this may be provided by adapting a displaced-plasma powder-coating gun. Such apparatus comprises corona discharge electrodes that are used to create a volume of ionised gas. This volume of ionised gas forms in the space between the electrodes but is displaced from this location between the electrodes by a flow of gas travelling through the volume of space between the electrodes. The gas-entrained particles can be introduced into this volume of ionised gas and thereby become corona-discharge activated.

Corona-discharge treating the particles using a displaced volume of ionised gas in this way has the advantage that there is less heating of the particles as they are treated. Depending on the material of the particle this allows them to keep their form better, by, for example, being less susceptible to fusion or melting. In addition, displacing the volume of gas used to treat the particles means that they can be treated without having to travel between the electrodes. Particles travelling between the electrodes will often deviate and become fused to the electrode, thus destroying the particle and reducing the efficiency of the electrode. The apparatus of these and other embodiments avoids this disadvantage. Also the flow of gas used to displace the volume of ionised gas from between the electrodes can be the same flow of gas that is used to entrain the particles. Thus the operation of the apparatus can be simplified and a correspondingly lower amount of gas can be used.

The corona discharge produced from the electrodes may be constant but is preferably pulsed. Pulsed-field corona discharge has the advantage that there is less heating of the particles during treatment and thus the risk of melting or fusing the treated particles is reduced.

Phage-containing droplets of liquid of a defined size may be produced by spraying from a nozzle. These droplets can then be entrained in a gas stream and treated by corona discharge in the same way as the particles described above.

In particular embodiments, the droplets are produced by passing the phage-containing solution through the nozzle of an electrostatic sprayer. This has the advantage of simultaneously producing a spray of droplets of defined size and electrostatically (corona-discharge) activating these droplets before entraining them in a gas stream. Thus the droplets to be combined with the particles are produced simply, accurately and efficiently by the use of this apparatus.

The apparatus also contains a chamber or space in which the gas streams are combined to bring the corona-discharge treated particles and droplets together. Once the reactions linking the phage to the particles are complete the length of the gas stream is adjusted to carry the phage-linked particles far enough that the liquid from the droplets is removed from the particles by evaporation while they are still entrained in the stream of gas and before reaching the collection area. Alternatively, the liquid can be removed form the particles following collection.

Apparatus in Series

Apparatus for manufacture of particles with phage covalently attached may be set up in series. In one example, the activated particle product from a reaction chamber is transferred to a downstream chamber for attachment of bacteriophage. In another the order is reversed: the bacteriophage droplet product from a reaction chamber is transferred to a downstream chamber for attachment to activated particles. A similar arrangement may be applied to activate a filament: filamentous material is introduced into a reaction chamber by continuous spooling such that activation, e.g. by corona discharge, occurs immediately preceding introduction of bacteriophage.

Substrate in Solution

When the polymer or other substrate is in solution a preferred embodiment comprises a three stage process whereby the initial charged droplets are gas dried and passed through a corona field with the same polarity as the initial droplet producing field, and mixed with the droplets of a bacteriophage suspension at a size that gives the desired number of bacteriophages bound per particle and with the opposite charge. This is followed by drying and collection of the neutral final product.

Substrate Free of Solvent

When the polymer or other material in the initial particle production is in melted form (not solution) an embodiment comprises an electrospray field in the corona producing voltage range to make particles that are mixed with particles of opposite charge from bacteriophage or other suspensions. In embodiments, the bacteriophage or other particles are dried before combination with the particles of polymer. In general, the solvent for the bacteriophage suspension may be water, an aqueous organic solvent or a liquid gas such as liquid CO₂.

Particle Preparation and Spraying

Conventional mechanical spraying systems and electro-spraying can be used for particle or fine particle production for use in the invention, based on controlled emergence from a nozzle of a droplet. Each droplet comprises material suspended in solution that will form the desired particle. As droplet liquid evaporates, the fine powder suspended within in it forms a tight cluster. For a droplet produced from a solution, the remaining substance tends to crystallise forming a solid particle and the size of such particles can be controlled by changing the concentration of dissolved or suspended substance.

Electrospraying (electrohydrodynamic spraying) is a method of liquid atomization by means of electrical forces. In electro-spraying, the liquid at the outlet of a nozzle is subjected to an electrical shear stress by maintaining the nozzle at high electric potential (e.g. 3-30 kV). An advantage of electro-spraying is that droplets can be extremely small and the charge and size of the droplets can be controlled by adjusting the flow rate and voltage applied to the nozzle. Moreover electro-spraying has additional advantages over conventional mechanical spraying systems where droplets are charged by induction: (1) droplets have size smaller than those available from conventional mechanical atomisers, and can be smaller than 1 μm; (2) the size distribution of the droplets is usually narrow, with low standard deviation; (3) charged droplets are self-dispersing in the space; and (4) the motion of charged droplets can be easily controlled (including deflection or focusing) by electric fields.

Electrospraying processes have been reviewed and summarised by Hayati et al. [1,2], Cloupeau and Prunet-Foch [3,4], Grace and Marijnisen [5], and Jaworek and Krupa [6,7]. Schultze [8], Shorey and Michelson [9], Mutoh et al. [10], and Smith [11] determined for liquids the range of physical parameters (mainly the values of its electrical conductivity) in which the liquid can be atomised by electrical forces.

For use in the invention, the process of solid particle production by electro-spraying is suitable with regards to the size of the droplets generated at given conditions and the frequency of their emission. Droplets can also be charged during the process of their atomisation by mechanical forces in the presence of electric field. Droplets generated by electro-spraying in the cone-jet mode can be as small as 1 μm, and for water droplet of this size, the specific charge can be 14 C/kg. Although the droplets generated mechanically with charging by induction are charged to a level one order of magnitude less than those produced by electro-spraying, the mechanical atomisation method may be used when large quantities of liquid are used. When droplets of controlled size are required, a synchronous excitation of the liquid jet either by mechanical or electrical methods can be used. A piezoelectric transducer placed in the liquid container, close to the nozzle outlet can be employed for the jet excitation. For mechanical methods of jet excitation, the application of pulsed or ac voltage superimposed onto dc bias voltage can, by controlling both the ac frequency and liquid volume flow rate, control droplet size and produce droplets of the required mean size. Fine particle generation is also possible by solvent evaporation from the droplets generated by electrospraying.

In operation of specific methods and apparatus of the invention, an initial action of corona activation fields is to produce free radicals on the material surface, followed by rapid decay into more stable hydrophilic groups. In order to produce covalent bonds between a particle and a bacteriophage the invention enables bringing the bacteriophage into contact with the treated surface rapidly (typically in less than a second) so that free radical based reactions can take place leading to covalent bond formation. Whereas this has hitherto been achieved on film surfaces where bacteriophages in suspension can be rapidly applied to the film surface by a variety of means it is through the invention that this is now efficiently and controllably possible with powders and similar particles. The invention can be operated with reduced time to make and collect/guide the activated powder (particles) and bring it into contact with the bacteriophages (or other particles).

A Specific System of the Invention

A system of the invention comprises particles and a spray gun, to create a spray of charged particles, such as a compressed air sprayer, e.g. an electrostatic gun or a corona gun which imparts a charge, typically a positive charge, to the particles (referred to also as powder due to the particle size). The powder is usually contained in a hopper in the apparatus and passes through the electrostatic spray gun, which charges the particles on emission.

A high voltage generator, usually specified to deliver variable voltages between 30 & 100 kV, is generally situated in close proximity to the powder feed hopper and the spray equipment.

A typical electrostatic particle generating system thus comprises:
A powder hopper
A source of compressed air at controlled humidity to transport the powder from hopper to gun
A high voltage generator (typically 30-100 kV)
Powder application guns which may be:
   hand operated
   automatic, either static, reciprocating or wagging.
Optionally, a specially designed unit, to allow excess powder to be removed by an air stream to a recovery unit comprising:
A recovery unit may consist of:
   a cyclone unit
   bag or frame filters, or
   a combination of both.
A chamber for combination of charged particles with bacteriophage-containing droplet.

Immobilisation onto Nylon Particles

In use of a specific embodiment of the invention, employing apparatus described in more detail in an example below, powder particles of nylon 6 polymer were produced by an electrospray system and were activated by a positive corona discharge. The activated powder was immediately mixed with the output from a parallel electrospray device producing negatively charged droplets of bacteriophage suspension of approximately 50 microns diameter. Particles were dried in an air flow and tested for bacteriophage activity in a standard plaque assay—the assay confirmed active phage had been attached to the nylon particles.

Particular Embodiments

As is apparent from the above, the invention provides, inter alia, the following embodiments

1. A method of covalently attaching a bacteriophage to a substrate, comprising:
generating a plasma between two electrodes;
flowing a fluid between the electrodes to displace the plasma or a portion of the plasma to a displaced zone not between the electrodes; and
(i) introducing the substrate into plasma in the displaced zone without the substrate passing between the electrodes and combining the substrate with the bacteriophage, or (ii) introducing the bacteriophage into plasma in the displaced zone without the bacteriophage passing between the electrodes, and combining the bacteriophage with the substrate.

2. A method according to embodiment 1, comprising introducing substrate into plasma in the displaced zone to activate the substrate and combining activated substrate with bacteriophage to yield bacteriophage covalently attached to the substrate.

3. A method according to embodiment 1, comprising introducing bacteriophage into plasma in the displaced zone to activate the bacteriophage and combining activated bacteriophage with substrate to yield bacteriophage covalently attached to the substrate.

4. A method according to embodiment 1, comprising introducing both bacteriophage and substrate into plasma in the displaced zone to activate both and combining activated substrate with activated bacteriophage to yield bacteriophage covalently attached to the substrate.

5. A method according to any previous embodiment, comprising holding the substrate stationary and moving the displaced zone relative to the substrate.

6. A method according to embodiment 5, comprising moving the displaced zone across a surface of the substrate.

7. A method according to embodiment 6, comprising applying bacteriophage to the surface and moving the displaced zone across the surface of the substrate to which bacteriophage has been applied.

8. A method according to any previous embodiment, wherein the fluid is a gas.

9. A method according to any previous embodiment, wherein the plasma is formed by an electric discharge between the electrodes.

10. A method according to embodiment 9, wherein the discharge is a corona discharge.

11. A method according to embodiment 9 or 10, wherein the discharge is a pulsed field corona discharge.

12. A method according to any previous embodiment, wherein the substrate comprises 0particles.

13. A method according to any previous embodiment, wherein the bacteriophage are provided as a suspension in liquid droplets.

14. A method according to embodiment 13, wherein the droplets are of diameter 150 microns or less.

15. Apparatus for carrying out the method of any of embodiments 1 to 14.

16. Apparatus according to embodiment 15, for covalently attached bacteriophage to a substrate, comprising:
(a) first and second electrodes and a plasma generator for producing a plasma between the electrodes;
(b) a first conduit connected to a supply of fluid under pressure for introducing a fluid flow between the electrodes, wherein flow of the fluid displaces the plasma or a portion of the plasma to a displaced zone located not between the electrodes;
(c) a second conduit connected to a supply of bacteriophage or substrate for introducing the bacteriophage or the substrate into the displaced zone without passing between the electrodes;
(d) a chamber in which bacteriophage and substrate are combined to covalently attach the bacteriophage to the substrate.

17. Apparatus according to embodiment 16, wherein the second conduit is for introducing bacteriophage into the displaced zone and further comprising a third conduit for introducing substrate into the displaced zone, so that bacteriophage and substrate are combined in the displaced zone.

18. Apparatus according to any of embodiments 15 to 17, comprising a plasma generator for generating a pulsed field corona discharge.

19. Apparatus according to any of embodiments 15 to 18, wherein the second conduit is connected to a supply of bacteriophage in suspension in a liquid for introducing the bacteriophage into the displaced zone as a suspension in liquid droplets.

20. A method of covalently attaching a bacteriophage to a substrate, comprising:
(a) combining (i) substrate with (ii) bacteriophage, wherein prior to or during the combining (i) or (ii) or both (i) and (ii) are activated, and wherein
(b) during the combining the bacteriophage is contained within a liquid droplet.

21. A method according to embodiment 20, wherein the droplet is of average diameter 150 microns or less.

22. A method according to embodiment 21, wherein the droplet is of average diameter 100 microns or less.

23. A method according to any previous embodiment, wherein the substrate comprises particles of average diameter 500 microns or less.

24. A method according to any previous embodiment, wherein the substrate comprises particles of average diameter 200 microns or less.

25. A method according to any of embodiments 20 to 24, wherein the liquid is aqueous.

26. A method according to embodiment 25, wherein the liquid is water.

27. A method according to any of embodiments 20 to 24, wherein the liquid is non-aqueous.

28. A method according to embodiment 27, wherein the liquid is or comprises a compound that is a gas at atmospheric pressures and 20° C. and wherein the method is carried out under conditions of temperature and pressure such that the compound forms liquid droplets.

29. A method according to embodiment 28 wherein the covalently attached bacteriophage and substrate product is subjected to modified conditions of temperature and/or pressure so that the compound evaporates, yielding dry product.

30. A method according to any of embodiments 20 to 29, for attachment of bacteriophage to a water-sensitive substrate.

31. A method according to embodiment 30, wherein the substrate comprises a superabsorbent polymer.

32. A method according to any of embodiments 20 to 31, comprising activating the substrate and combining activated substrate with the liquid droplet.

33. A method according to any of embodiments 20 to 32, comprising activating the droplet and combining the activated droplet with the substrate.

34. A method according to any of embodiments 20 to 33, comprising activating both substrate and droplet and combining them

35. A method according to any of embodiments 20 to 34, comprising combining droplets of a predetermined size prepared from a suspension of bacteriophage of predetermined concentration so as to control the number and/or density of bacteriophage attached to the substrate.

36. A method according to any of embodiments 20 to 35, comprising combining droplets of mass median diameter 1-200 microns with particles of mass media diameter 1-200 microns.

37. A method according to embodiment 36, comprising combining droplets of diameter 100 microns or less with particles of diameter 100 microns or less.

38. A method according to embodiment 36 or 37 wherein the ratio of the respective diameter of droplets and particles is from 1:3-3:1.

39. A method according any of embodiments 20 to 38, comprising activating both particles and substrate, wherein the particles are activated using an electric discharge and the substrate is activated using an oppositely charged electric discharge.

40. A method according to embodiment 39, wherein the discharge is a corona discharge.

41. A method according to embodiment 40, comprising activating the particles using a negatively charged corona discharge and activating the droplets using a positively charged corona discharge.

42. A method according to any of embodiments 20 to 41, comprising simultaneously forming and activating the particles.

43. Apparatus for carrying out the method of any of embodiments 20 to 42.

44. Apparatus according to embodiment 43, for covalently attaching bacteriophage to a substrate, comprising:
(a) means to generate droplets containing bacteriophage;
(b) (i) a plasma generator for generating a plasma in combination with a droplet activating station to contact the droplets of (a) with the plasma, or (ii) a plasma generator for generating a plasma in combination with a substrate activating station to contact the substrate with the plasma; and
(c) a chamber in which substrate and bacteriophage can be combined at the same time as or after contact with the plasma so as to form a covalent bond between the bacteriophage and the substrate.

45. Apparatus according to embodiment 44, adapted to generate liquid droplets of a solvent or solution that is a gas at room temperature and 20° C. containing bacteriophage.

46. Apparatus according to embodiment 44 or 45, wherein the plasma generator of b (ii) is adapted for generating a plasma to activate the substrate.

47. Apparatus according to embodiment 44 or 45, wherein the plasma generator of b (i) is adapted for generating a plasma to activate the droplets containing bacteriophage.

48. Apparatus according to embodiment 44 or 45, wherein the plasma generator and the chamber are arranged so that the bacteriophage and the substrate are combined and contacted with the plasma so as to be activated at the same time and in the same chamber.

49. Apparatus according to any of embodiments 44 to 48, wherein the substrate comprises particles of average diameter 500 microns or less.

50. A method of covalently attached two bacteriophage of different strain or type to a single substrate particle, comprising:
(a) combining the particle with a first bacteriophage, wherein prior to or during the combining the particle or the bacteriophage or both are activated, so as to yield an intermediate product comprising a particle to which the first bacteriophage is covalently attached; and
(b) combining the intermediate product of (a) with a second bacteriophage of different strain or type to the first, wherein prior to or during the combining the intermediate product or the second bacteriophage or both are activated, so as to yield a product comprising a particle to which the first and second bacteriophage are covalently attached.

51. A method according to embodiment 50, comprising combining the product with a third bacteriophage of different strain or type to the first and second, wherein prior to or during the combining the product or the third bacteriophage or both are activated, so as to yield a further product comprising a particle to which the first, second and third bacteriophage are covalently attached.

52. A method according to embodiment 50 or 51, wherein substrate or bacteriophage or intermediate product is activated in steps a and b by an electric discharge.

53. A composition comprising a plurality of particles to which first and second bacteriophage of different strain or type are covalently attached, wherein at least 50% of the particles by number comprise at least one first bacteriophage and at least one second bacteriophage.

54. A composition according to embodiment 53, wherein at least 60% of the particles by number comprise at least one first bacteriophage and at least one second bacteriophage.

55. A composition according to embodiment 53, wherein at least 70% of the particles by number comprise at least one first bacteriophage and at least one second bacteriophage.

The invention is now described with reference to the accompanying drawings, in which:

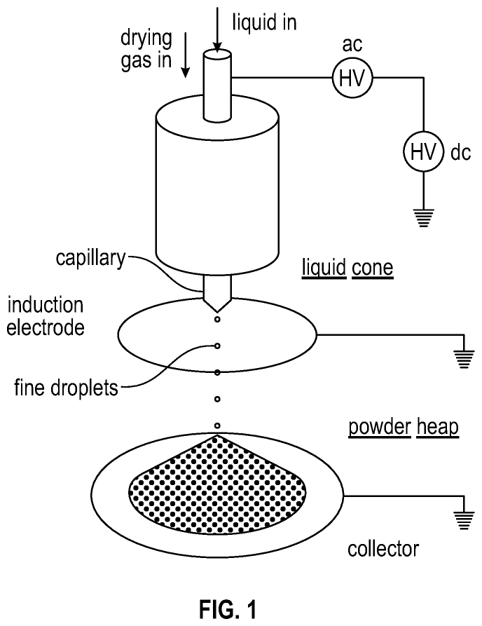

FIG. 1 shows a schematic diagram of apparatus for use in the invention for generation of activated polymer particles;

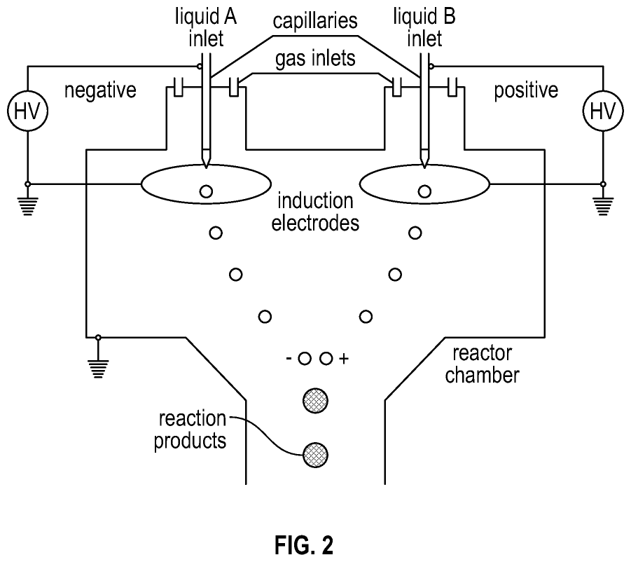

FIG. 2 shows a schematic diagram of apparatus of the invention for production of immobilised bacteriophage; and

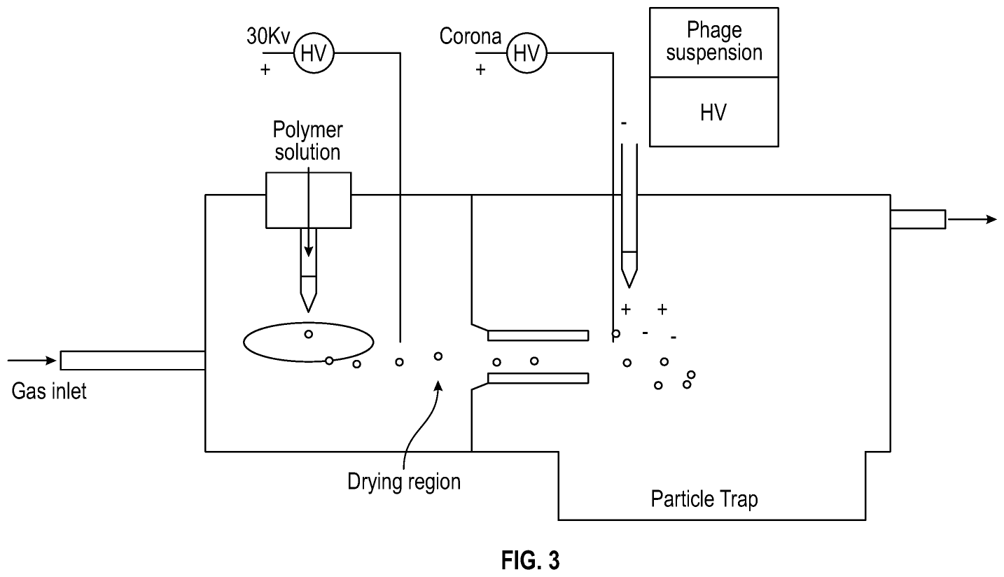

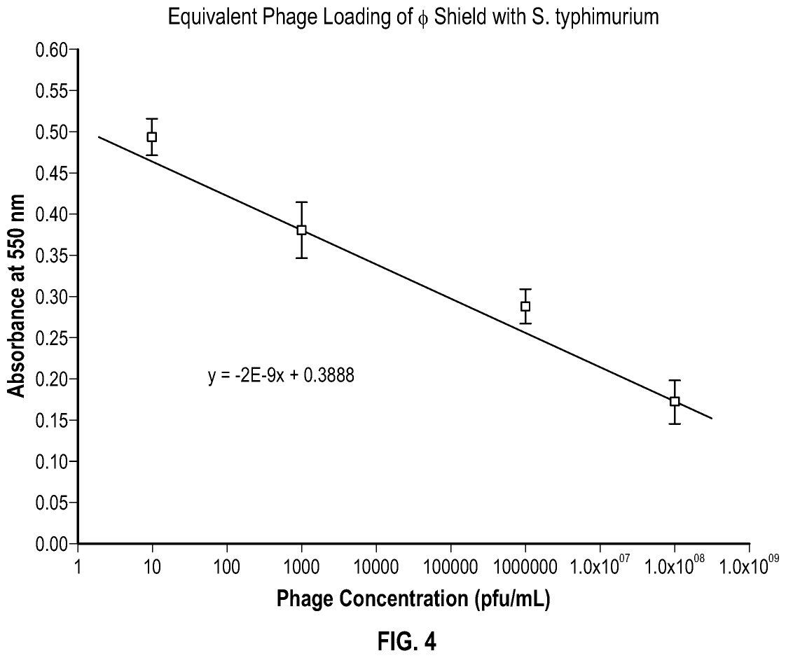

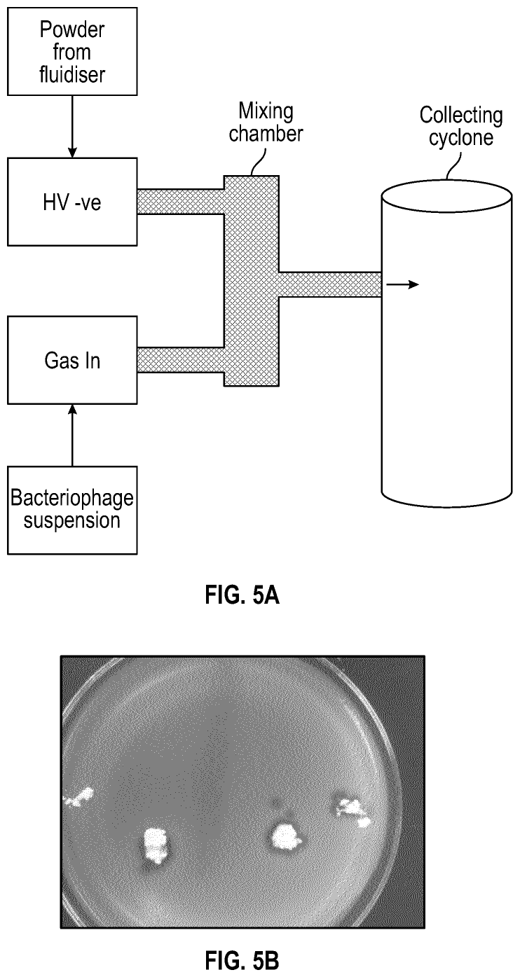

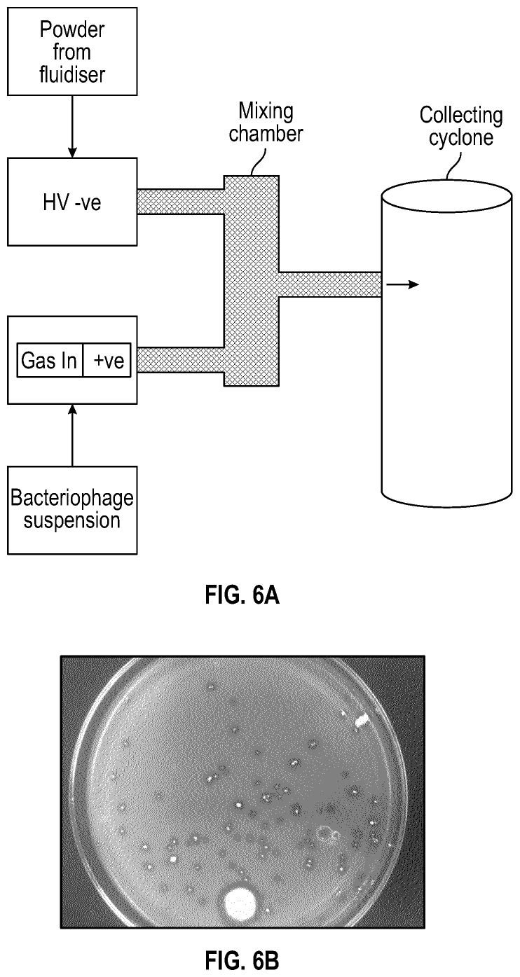

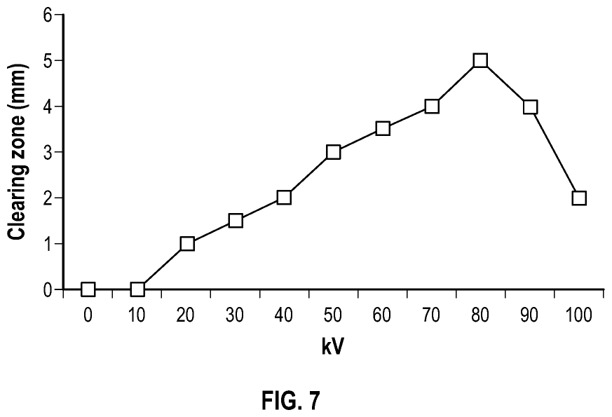

FIG. 3 shows a schematic diagram of further apparatus of the invention for production of immobilised bacteriophage.

### EXAMPLE 1

Apparatus was designed for immobilisation of bacteriophage (and other molecules) onto the activated surface of particles and filaments for the manufacture of bulk product. The apparatus was designed to permit the corona activation of materials, particularly particles, and reaction with bacteriophages or other viruses and substances to take place very rapidly, and within the lifetime of the induced free radicals.

Referring to FIG. 1, this shows a basic electrospray system comprising a high voltage supply to produce a corona between the induction electrode and the liquid nozzle, producing in operation a stream of polymer particles activated and ready for covalent attachment to bacteriophage.

FIG. 2 shows how the particle activator of FIG. 1 is integrated with a second liquid intake having opposite polarity.

Via inlet A is introduced a bacteriophage suspension at a suitable concentration and sufficient electric field so that emerging droplets are negatively charged. In parallel, via inlet B is introduced a polymer solution to be discharged through an electro-spray nozzle to form positively charged droplets—in proximity to the bacteriophage containing droplets.

In operation, particle surfaces are activated by corona discharge upon emergence from the electro-spray nozzles and combination with oppositely charged particles/droplets and immobilisation (covalent attachment) occurs within the reaction chamber. Through the reaction chamber, flow of drying gas facilitates transport and collection of particles.

### EXAMPLE 2

A second apparatus for immobilisation of bacteriophage onto particles was similarly designed to permit the corona activation of materials, particularly particles, and reaction with bacteriophages or other viruses and substances to take place very rapidly, and within the lifetime of the induced free radicals.

In the second apparatus, shown schematically in FIG. 3, particle production using an electro-spray system is combined with a secondary corona stage.

The secondary corona stage has the same polarity and is situated to take advantage of particle flow using inert gas. A second spray nozzle is employed for bacteriophage droplet production with an opposite polarity and introduction of the bacteriophage droplets into the mixing chamber. Mixing of the charged bacteriophage droplets with the opposite charged polymer droplets results in rapid contact and combination in less than a second, leading to covalent attachments being formed.

### REFERENCES

[1] I. Hayati, A. I. Bailey, T. F. Tadros, Investigations into the mechanisms of electrohydrodynamic spraying of liquids. Pt. I. Effect of electric field and the environment on pendant drop and factors affecting the formation of stable jets and atomisation, J. Colloid Interface Sci. 117 (1) (1987), pp 205-221.

[2] I. Hayati, A. I. Bailey, T.F. Tadros, Investigations into the mechanisms of electrohydrodynamic spraying of liquids. Pt. II. Mechanism of stable jet formation and electrical forces acting on a liquid cone, J. Colloid Interface Sci. 117 (1) (1987), pp 222-230.

[3] M. Cloupeau, B. Prunet-Foch, Electrostatic spraying of liquids, Main functioning modes, J. Electrostat. 25 (1990), pp 165-184.

[4] M. Cloupeau, B. Prunet-Foch, Electrohydrodynamic spraying functioning modes. A critical review, J. Aerosol Sci. 25 (6) (1994), pp 1121-1136.

[5] J. M. Grace, J. C. M. Marijnissen, A review of liquid atomization by electrical means, J. Aerosol Sci. 25 (6) (1994), pp 1005-1019.

[6] A. Jaworek, A. Krupa, Jet and drop formation in electrohydrodynamic spraying of liquids. A systematic approach, Exp. Fluids 27 (1) (1999), pp 43-52.

[7] A. Jaworek, A. Krupa, Classification of the modes of EHD spraying, J. Aerosol Sci. 30 (7) (1999), pp 873-893.

[8] K. Schultze, Das Verhalten verschiedener Flussigkeiten bei der Elektrostatischen Zerstaubung, Z. Angew. Phys. 13 (1) (1961), pp 11-16.

[9] J. D. Shorey, D. Michelson, On the mechanism of electrospraying, Nucl. Instrum. Methods 82 (1970), pp 295-296.

[10] M. Mutoh, S. Kaieda, K. Kamimura, Convergence and disintegration of liquid jets induced by an electrostatic field, J. Appl. Phys. 50 (5) (1979), pp 3174-3179.

[11] D. P. H. Smith, The electrohydrodynamic atomization of liquids, IEEE Trans. Ind. Appl. 22 (3) (1986), pp 527-535.

1. A method of covalently attaching a bacteriophage to a substrate, comprising:
(a) combining (i) substrate with (ii) bacteriophage, wherein prior to or during the combining (i) or (ii) or both (i) and (ii) are activated, and wherein
(b) during the combining the bacteriophage is contained within a liquid droplet.

2-3. (canceled)

4. A method according to claim 1, wherein the substrate comprises particles of average diameter 500 microns or less.

5. A method according to claim 1, wherein the substrate comprises particles of average diameter 200 microns or less.

6. A method according to claim 1, wherein the liquid is aqueous.

7. A method according to claim 6, wherein the liquid is water.

8. A method according to claim 1, wherein the liquid is non-aqueous.

9. A method according to claim 8, wherein the liquid is or comprises a compound that is a gas at atmospheric pressures and 20 oC. and wherein the method is carried out under conditions of temperature and pressure such that the compound forms liquid droplets.

10. A method according to claim 9 wherein the covalently attached bacteriophage and substrate product is subjected to modified conditions of temperature and/or pressure so that the compound evaporates, yielding dry product.

11. A method according to claim 1, for attachment of bacteriophage to a water-sensitive substrate.

12. A method according to claim 11, wherein the substrate comprises a superabsorbent polymer.

13. A method according to claim 1, comprising activating the substrate and combining activated substrate with the liquid droplet.

14. A method according to claim 1, comprising activating the droplet and combining the activated droplet with the substrate.

15. A method according to claim 1, comprising activating both substrate and droplet and combining them

16. (canceled)

17. A method according to claim 1, comprising combining droplets of mass median diameter 1-200 microns with particles of mass media diameter 1-200 microns.

18. A method according to claim 17, comprising combining droplets of diameter 100 microns or less with particles of diameter 100 microns or less.

19. A method according to claim 17, wherein the ratio of the respective diameter of droplets and particles is from 1:3-3:1.

20-24. (canceled)

25. Apparatus for covalently attaching bacteriophage to a substrate according to the method of claim 1, comprising:
a) means to generate droplets containing bacteriophage;
b) (i) a plasma generator for generating a plasma in combination with a droplet activating station to contact the droplets of (a) with the plasma, or (ii) a plasma generator for generating a plasma in combination with a substrate activating station to contact the substrate with the plasma; and
c) a chamber in which substrate and bacteriophage can be combined at the same time as or after contact with the plasma so as to form a covalent bond between the bacteriophage and the substrate.

26. Apparatus according to claim 25, adapted to generate liquid droplets of a solvent or solution that is a gas at room temperature and 20 oC. containing bacteriophage.

27. Apparatus according to claim 25, wherein the plasma generator of b (ii) is adapted for generating a plasma to activate the substrate.

28. Apparatus according to claim 25, wherein the plasma generator of b (i) is adapted for generating a plasma to activate the droplets containing bacteriophage.

29.-55. (canceled)
